(19) United States
(12) Patent Application Publication
(10) Pub. No.: US 2024/0415659 A1
(43) Pub. Date: Dec. 19, 2024

(54) IMPLANTABLE MEDICAL DEVICE WITH FLUIDICS PUMP

(71) Applicant: Boston Scientific Scimed, Inc., Maple Grove, MN (US)

(72) Inventors: Jean M. Bobgan, Maple Grove, MN (US); Derek John Boettger, Woodbury, MN (US); Matthew William Couri, Plymouth, MN (US); Robert Hannon, Clonmel (IE); Thomas Sinnott, Enniscorthy (IE); Richard Percy, Leamlara (IE)

(21) Appl. No.: 18/733,392

(22) Filed: Jun. 4, 2024

Related U.S. Application Data
(60) Provisional application No. 63/508,779, filed on Jun. 16, 2023.

(51) Int. Cl. A61F 2/26

(52) U.S. Cl. CPC A61F 2/26; A61F 2220/0025; A61F 2240/001; A61F 2250/0001; A61F 2250/0078

(57) ABSTRACT

An implantable medical device is disclosed. The implantable medical device includes a housing forming an internal compartment. The housing includes a frame disposed within the internal compartment forming a first partition and a second partition within the internal compartment. The first partition is hermetically sealed from the second partition. At least one electronic component and a power source are disposed within the first partition. The medical device includes a fluidics circuit including a manifold operably coupled to at least one fluidics component. The fluidics circuit is operably coupled to the at least one electronic component across the frame.

### CROSS-REFERENCE TO RELATED APPLICATION

This application claims priority to U.S. Provisional Patent Application No. 63/508,779, filed on Jun. 16, 2023, entitled “IMPLANTABLE MEDICAL DEVICE WITH FLUIDICS PUMP”, the disclosure of which is incorporated by reference herein in its entirety.

### TECHNICAL FIELD

The present disclosure relates generally to medical systems and implantable medical devices. More specifically, the present disclosure relates to implantable medical devices including a fluidics pump.

### BACKGROUND

Implantable medical devices include electrical or electromechanical medical devices that are implanted within a patient and perform a task such as to monitor a parameter of the patient or to deliver a therapy to the patient via electrical energy. For example, the implantable medical device can be an implantable urological device implanted into the patient to treat a condition such as erectile disfunction, penile deformity, or incontinence as an implantable fluid operated inflatable device. Implantable fluid operated inflatable devices often include one or more pumps that regulate a flow of fluid between different portions of the implantable device to provide for inflation and deflation of a fluid fillable implant component of the device. A valve can be positioned within fluid passageways of the device to direct and control the flow of fluid to achieve inflation, deflation, pressurization, depressurization, activation, or deactivation of the different fluid fillable implant components of the device. In some implantable fluid operated inflatable devices, sensors can be used to monitor fluid pressure, fluid volume, or fluid flow rate within fluid passageways of the device.

### SUMMARY

In Example 1, an implantable medical device comprising a housing forming an internal compartment. The housing includes a frame disposed within the internal compartment forming a first partition and a second partition within the internal compartment. The first partition is hermetically sealed from the second partition. At least one electronic component and a power source are disposed within the first partition. The medical device includes fluidics circuit including a manifold operably coupled to at least one fluidics component. The fluidics circuit is operably coupled to the at least one electronic component across the frame.

In Example 2, the implantable medical device of Example 1, wherein the implantable medical device is included in an implantable urological device.

In Example 3, the implantable medical device of Example 2, wherein the implantable urological device is an inflatable penile prosthesis.

In Example 4, the implantable medical device of any of Examples 2-3, wherein the implantable urological device includes a reservoir having a fluid, and the implantable medical device is in fluid communication with the reservoir.

In Example 5, the implantable medical device of Example 4, wherein the implantable urological device includes a plurality of inflatable cylinders to receive the fluid, and the implantable medical device is in fluid communication with the plurality of inflatable cylinders, and wherein the implantable medical device is configured to pump the fluid from the reservoir to the plurality of inflatable cylinders.

In Example 6, the implantable medical device of any of Examples 1-5, wherein the implantable medical device is included in a medical system further comprising a remote charger and a remote programmer.

In Example 7, the implantable medical device of Example 6 wherein the remote charger is operably coupleable to an interface system to provide transcutaneous inductive power transfer to a secondary conductor.

In Example 8, the implantable medical device of any of Examples 6-7, wherein the programmer is a handheld programmer in radiofrequency communication with the communication system via an antenna.

In Example 9, the implantable medical device of Example 8, wherein the programmer includes a software application operating on a mobile computing device to operate the implantable medical device.

In Example 10, the implantable medical device of any of Examples 1-9, wherein the fluidics circuit is operably coupled to the at least one electronic component across the frame via a hermetically sealed feedthrough connection.

In Example 11, the implantable medical device of any of Examples 1-10, wherein the manifold is integrally formed with the frame.

In Example 12, the implantable medical device of Example 11, wherein the at least one electronic component is coupled to the manifold in the first partition.

In Example 13, the implantable medical device of Examples 1-12, wherein the fluidics components are arranged in a planar configuration.

In Example 14, the implantable medical device of Example 12, wherein the fluidics components include two piezoelectric pumps in a single fluid channel.

In Example 15, the implantable medical device of Example 14, wherein the fluidics components further include a valve and a pressure sensor in the single fluid channel.

In Example 16, an implantable medical device. The implantable medical device comprises a housing forming an internal compartment. The housing includes a frame disposed within the internal compartment forming a first partition and a second partition within the internal compartment. The first partition is hermetically sealed from the second partition. At least one electronic component and a power source are disposed within the first partition. The medical device includes a fluidics circuit including a manifold operably coupled to at least one fluidics component. The fluidics circuit is operably coupled to the at least one electronic component across the frame.

In Example 17, the implantable medical device of Example 16, wherein the power source is a rechargeable power source including a battery.

In Example 18, the implantable medical device of Example 17, wherein the electronic components include a communication system and a recharge system, the recharge system coupled to the power source.

In Example 19, the implantable medical device of Example 18, and further comprising an antenna operably coupled to the communication system and a secondary coil operably coupled to the recharge system.

In Example 20, the implantable medical device of Example 19 wherein the antenna and secondary coil are disposed outside of the internal compartment, wherein a header is operably coupled to the housing outside of the internal compartment and forming an internal region, and the antenna and secondary coil are disposed within the internal region.

In Example 21, the implantable medical device of Example 16, wherein the manifold is integrally formed with the frame.

In Example 22, the implantable medical device of Example 21, wherein the manifold is hermetically sealed to the housing to form the first partition and the second partition.

In Example 23, the implantable medical device of Example 22, wherein the at least one electronic component is coupled to the manifold in the first partition.

In Example 24, the implantable medical device of Example 16, wherein the fluidics circuit is operably coupled to the at least one electronic component across the frame via a hermetically sealed feedthrough connection.

In Example 25, the implantable medical device of Example 16, wherein the fluidics component ac arranged in a planar configuration.

In Example 26, the implantable medical device of Example 25, wherein the fluidics components include two piezoelectric pumps in a single fluid channel.

In Example 27, the implantable medical device of claim of Example 26, wherein the fluidics components further include a valve and a pressure sensor in the single fluid channel.

In Example 28, the implantable medical device of Example 16, wherein the fluidics components are arranged in a stacked configuration.

In Example 29, a method of manufacturing an implantable medical system. A frame including a fluidics manifold is formed. The fluidics manifold includes a first major surface and a second major surface. At least one electronic component and a power source are operably coupled to the first major surface. At least one fluidics component is operably coupled to the second major surface to form a fluidic circuit. A housing is attached to the frame to form an internal compartment, wherein the frame defines a first partition of the internal compartment and a second partition of the internal compartment, the first partition hermetically sealed from the second partition. The at least one electronic component is operably coupled to the fluidic circuit across the frame.

In Example 30, the method of Example 29, and further comprising forming a passageway within the fluidics manifold and chambers in the second major surface, the chambers fluidically coupled to passageway, wherein the at least one fluidics component is operably coupled to the chambers.

In Example 31, the method of Example 29, wherein the manifold is integrally formed with the frame.

In Example 32, an implantable urological device comprising a reservoir configured to receive a fluid, an inflatable member in fluid communication with the reservoir, and an implantable medical device. The implantable medical device includes a housing forming an internal compartment. The housing includes a frame disposed within the internal compartment forming a first partition and a second partition within the internal compartment. The first partition is hermetically sealed from the second partition. At least one electronic component and a power source are disposed within the first partition. The medical device includes a fluidics circuit including a manifold operably coupled to at least one fluidics component. The fluidics circuit is operably coupled to the at least one electronic component across the frame.

In Example 33, the implantable urological device of Example 32, wherein the inflatable member includes a plurality of inflatable cylinders in fluid communication with the medical actuation device and the reservoir, the plurality of inflatable cylinders configured to be disposed within a corpora cavernosa of a penis.

In Example 34, the implantable urological device of Example 32, wherein the medical instrument is configured to be disposed within a retropubic space.

In Example 35, the implantable urological device of Example 32, wherein the manifold is integrally formed with the frame.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example implantable medical system including an example implantable medical device, an example external charger, and an example programmer of the present disclosure.

FIG. 2 is a schematic diagram illustrating an example implantable urologic device including features of the example implantable medical device of FIG. 1.

FIG. 3A is a schematic diagram illustrating an example fluid architecture of the example implantable urologic device of FIG. 2.

FIG. 3B is a schematic diagram illustrating another example fluid architecture of the example implantable urologic device of FIG. 2.

FIG. 4A is a perspective view illustrating an example feature of the example implantable urologic device of FIG. 2.

FIG. 4B is a perspective view illustrating the example feature of FIG. 4A including structures in phantom.

FIG. 4C is a perspective view illustrating an example feature of the example implantable urologic device of FIG. 2 including structures in phantom.

FIGS. 5A-5C are schematic diagrams illustrating an example operation of a piezoelectric pump assembly for use with the example implantable urologic device of FIG. 2.

FIGS. 6A-6C are schematic diagrams illustrating an example operation of another piezoelectric pump assembly for use with the example implantable urologic device of FIG. 2.

FIG. 7 is an exploded view of an example feature of the example implantable urologic device of FIG. 2.

FIG. 8 is a perspective view of another example feature of the example implantable urologic device of FIG. 2.

FIG. 9 is an exploded view of the example feature of FIG. 8.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

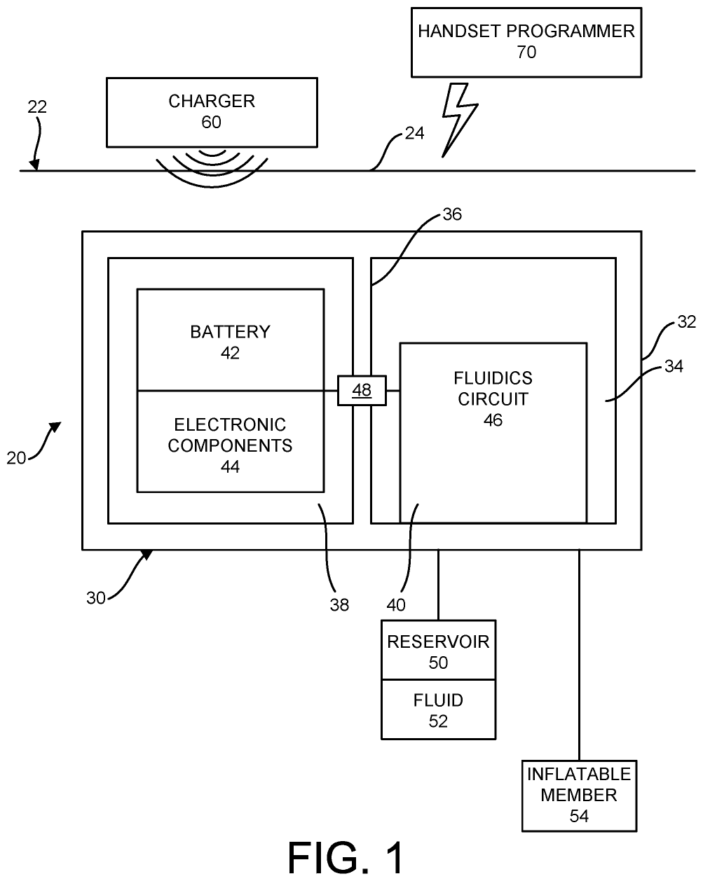

FIG. 1 illustrates an implantable medical device system 20. The implantable medical system 20 includes an implantable medical device 30, which can be fully implanted within a patient 22. The implantable medical device 30 can include a housing 32 forming an internal compartment 34. The housing 32 includes a frame 36 within the internal compartment 34 forming a first partition 38 and a second partition 40. The first partition 38 is hermetically scaled from the second partition 40. The implantable medical device 30 can include an energy storage system, such as a rechargeable power source 42 or a rechargeable battery, and electronic components 44 within the first partition 38. A fluidics circuit 46 is included within the second partition 40. The fluidics circuit 46 is operably coupled to the electronic components 44 across the frame 36 such as via a hermetic interface 48. In the illustrated example, the rechargeable power source 42 and electronic components 44 can be coupled to structures such as coils and antennae that may receive and transmit communication signals and receive power signals.

The implantable medical device 30 can include or be coupled to a reservoir 50 filled with a fluid 52, such as a sterile saline solution, in fluid communication with the fluidics circuit 46. Further, the implantable medical device 30 can include or be coupled to an inflatable member 54 in fluid communication with the fluidics circuit 46 and the reservoir 50 to receive the fluid. The electronic components 44, powered by the rechargeable power source 42, can provide for the monitoring and control of various operations of the fluidics circuit 46. The fluidics circuit 46 can provide for the transfer of fluid 52 between the reservoir 50 and the inflatable member 52. In one example, the fluidics circuit 46 can include a manifold, and fluidics components such as a pump assembly, a valve assembly, and a pressure sensor. In one example, the electronic components 44 can include a recharge system, a communication system, and a control system.

The implantable medical system 20 also includes a charger 60, which can also be referred to as a wireless recharger, outside of the patient 22, or across transcutaneous boundary 24 such as the surface of the patient's skin proximate the implantable medical device 30 to provide power signals and, in some examples, communication to the medical device 30. In some examples, transcutaneous charging is performed via radiofrequency power transfer or transmission or inductive power transfer or transmission. In this example, the external charger 60 does not mechanically connect with the implantable medical device, and the external charger can be used to charge the implantable medical device 30 from a relatively short distance away. In one example, the charger 60 is placed against the patient 22 and proximate the implantable medical device 30 to inductively transfer energy and to replenish the battery of the implantable medical device 30.

In an example of a charger 60, inductive power transfer can be performed with an inductive coupling between conductors, which may be formed as coils of wire, such as a primary coil in the charger 60 and a secondary coil in the implantable medical device 30. Power is transferred between the coils with a magnetic field. An alternating current through the primary coil creates an oscillating magnetic field. The magnetic field passes through the secondary coil, and the magnetic field induces an alternating electromotive force, or EMF, such as voltage, which creates an alternating current in the secondary conductor. The induced alternating current may either directly drive a load in the implantable medical device 30 or be rectified to direct current by a rectifier in the implantable medical device 30, which drives the load. Resonant inductive coupling is a type of inductive coupling in which power is transferred by magnetic fields between two resonant circuits, one in the charger 60 and one in the implantable medical device 30. Each resonant circuit can include a coil of wire connected to a capacitor, or a self-resonant coil or other resonator with internal capacitance. Resonant circuits, or tank circuits, are tuned to resonate at generally the same resonant frequency. The resonance between the conductors can greatly increase coupling and power transfer between the charger 60 and the implantable medical device 30.

In the example, the charger 60 delivers magnetic energy to a corresponding implantable device 30 at the preselected frequency with a resonant inductor-capacitor (LC) tank circuit to generate an H-field. The tank circuit includes a recharge coil in series with a recharge capacitor. Various configurations of the charger 60 can share a common coil design, and the preselected recharge frequency is determined via a selected tank capacitance of the recharge capacitor. The tank circuit can oscillate at a resonant frequency. A phase locked loop in the tank circuit is created via pulsing an applied tank voltage in phase with a tank current. During resonance, the tank current is approximately or generally sinusoidal over time. The tank circuit can achieve maximum tank power when a tank voltage pulse is aligned in time with the tank current. Recharge power can be adjusted by altering the magnitude and duty of the tank voltage pulse input to the tank circuit.

The charger 60 is available in different configurations depending on recharge frequencies and communication schemes for use with the implantable medical device 30. For example, a first configuration of the charger 60 may support a bidirectional inductive telemetry communication scheme and a first recharge frequency, a second configuration of the charger 60 may support a radiofrequency telemetry and downlink inductive telemetry communication schemes and a second recharge frequency, and a third configuration of the charger may support the bidirectional inductive telemetry communication scheme and a third recharge frequency. Inductive charging is presented in this disclosure for illustration, and the charger 60 may use other forms of wireless charging to replenish the rechargeable power source.

System 20 can also include a handset programmer 70 configured to wirelessly interface and communicate with the implantable medical device 30 or with the charger 60. In one example, the handset programmer 70 can be implemented as a general-purpose computing device or mobile computing device that hosts a software application. For instance, the handset programmer 70 can include a set of controls to transcutaneously communicate with or operate the implantable medical device 30 via the communication system or to communicate with or operate the charger 60. The medical device 30 can include an antenna to receive and transmit communication signals with the handset programmer 70. For example, the handset programmer 70 and medical device 30 can communicate via radiofrequency signals in the 2.400-2.4835 GHZ range as in Bluetooth Low Energy communication in, for example, a wireless personal network. Additionally, or alternatively, the handset programmer 70 or charger 60 may apply other forms of wireless communication, which can include other forms of radiofrequency communication or inductive communication. The handset programmer 70 may be configured with a user interface, such as a graphical user interface, to receive commands such as via soft buttons to wirelessly operate the medical device 30, receive feedback or parameters form the medical device 30, update a control system on the medical device, and interface with a computer network to provide telemetry or to allow control of the medical device 30 by a remote, computer network-connected system. For example, a user can apply the handset programmer 70 to actuate the medical device 30 such that the fluid 52 is pumped from the reservoir 50 into the inflatable member 54 or to release fluid 52 from the inflatable member 54 to return to the reservoir 50.

Systems of the present disclosure can optionally include additional components. The system 20 can include a charging dock, which can be plugged into a wall outlet and configured to charge an internal battery of the charger 60. The charger 60 can also be used in conjunction with a fixation product of system 20 to keep the charger 60 in position proximate the implantable medical device 30 during a recharge session. The fixation product can include a fixation belt to be worn around a portion of the patient 22 such as the belt line for an implantable medical device 30 in the abdomen, back, buttocks, or flank of the patient 22, or a fixation drape to be worn around the neck with a counterweight to balance the charger 60 for an implantable medical device 30 in the pectoral region of the patient 22. The fixation product receives the charger 60 to hold the charger 32 in place with respect to the fixation product so that the charger 60, in one example, does not rotate and generally does not move with respect to the implantable medical device 30 during the recharge session and to secure the charger 60 so as not to fall out unless purposefully removed from the fixation product.

The implantable medical device 30 can be implanted subcutaneously in an implantation locations or pockets within a patient 22. In some examples, another component of the implantable medical device 30 can occupy the same or an additional location within the patient 22. The implantable medical device 30 may be configured to deliver therapy to a patient, monitor parameters within a patient, receive and deliver signals with a patient, such as at regular intervals, continuously, or in response to a detected event such as an event detected by sensors, received from another implantable device (not shown), or received from components of the implantable medical system 20 such as from charger 60 or from handset programmer 70. The implantable medical device 30 can be configured to detect a variety of physiological signals that may be used in connection with various diagnostics, therapeutic, and other monitoring implementations. The implantable medical device 30 can be used in urological, neurological, cardiac, and other applicable filed that apply implantable medical devices with power systems or for receiving and transmitting signals.

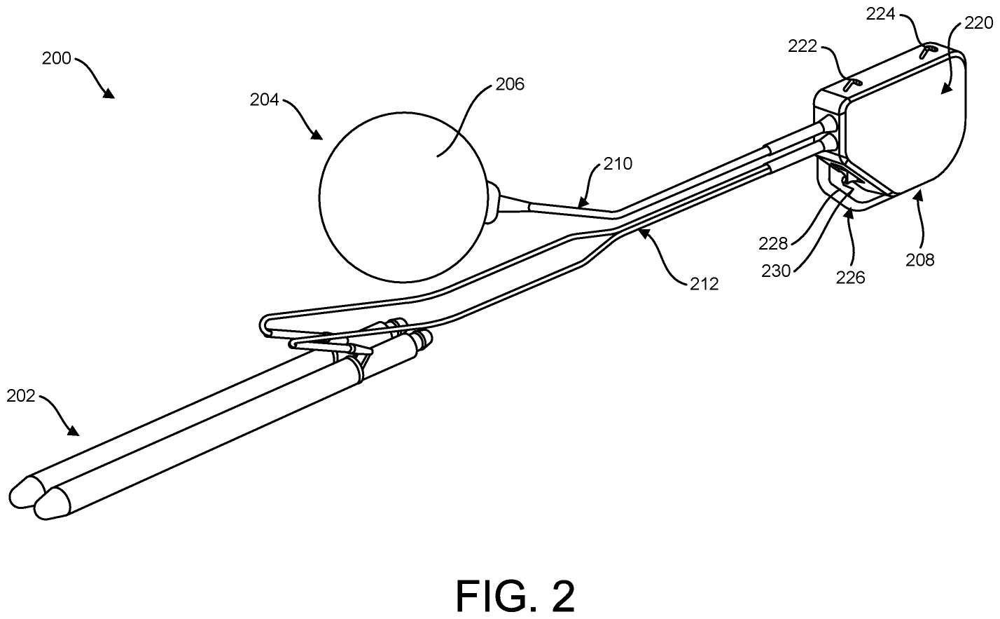

FIG. 2 illustrates an example implantable medical device 200 such as an implantable urologic device in connection with an inflatable penile prosthesis, which may correspond with implantable medical device 30. In the example implantable medical device 200 includes an inflatable member, such as a pair of inflatable cylinders 202, a reservoir 204 that may be filled with a fluid, such as a sterile saline solution 206, and an electromechanical actuation device 208 in a closed system. The reservoir 204 is fluidically coupled to the actuation device 208 via tubing 210, and the actuation device 208 is fluidically coupled to the cylinders 202 via tubing 212. For example, tubing 210, 212 can be kink-resistant tubing made from a silicone elastomer.

The actuation device 208 includes a hermetically sealed housing 220 formed of a biocompatible material such as titanium or steel. In one example, the housing 220 is formed via a plurality of walls welded together. The actuation device 208 includes an internal fluidics circuit to fluidically couple the reservoir 204 to the cylinders 202 via tubing 210, 212. Internal electronic components, powered by a rechargeable power source, can provide for the monitoring and control of various operations of the fluidics circuit such as the transfer of fluid 206 between the reservoir 204 and the cylinders 202. In one example, the electromechanical actuation device 208 can correspond with implantable medical device 30, the reservoir 204 and fluid 206 can correspond with reservoir 50 and fluid 52, respectively, and cylinders 202 can correspond with inflatable member 54 of FIG. 1.

The housing 220 forms an internal compartment defined by the space within the actuation device 208 that is bordered by the plurality of walls. The internal compartment of the actuation device 208 is configured to carry rechargeable energy storage system, electronics components, and a fluidic circuit. In one example, the internal compartment may include a plurality of partitions that can be separated by a frame and hermetically sealed from one another within the internal compartment. For example, the rechargeable energy storage system and electronics components may be carried in a first partition, and the fluidic circuit may be carried in a second partition. The first partition is hermetically sealed from the second partition. Feedthrough pins can be applied across the hermetic seal to allow the electronics components to control and monitor the fluidic circuit. In one example, the electronics components can be implemented by various components including resistors, capacitors, transistors, and integrated circuits disposed on one or more printed circuit boards within first partition of the internal compartment. The fluidic circuit can be implemented via titanium manifolds and electromagnetic pumps or piezoelectric pumps. In one example, the housing 220 can including an attachment device, such as loops 222, 224 formed a wall to receive sutures that can be applied to anchor the actuation device 208 to a structure within the patient.

The actuation device 208 can include a header 226 to form a region that includes power and communication interface structures such as a secondary coil 228 and an antenna 230. The header 226 is configured to allow the transmission of power and communication signals between the secondary coil 228 and the charger and between the antenna 230 and a handset programmer or charger. For example, the header 226 may be comprised of an overmolded polymer. The antenna and secondary coil are electrically coupled to the electronic components within the first partition, such as via a hermetic feedthrough component.

The cylinders 202 are typically implanted in the corpora cavernosa of the penis, and the reservoir 204 are often implanted in the retropubic space, or Retzius space, of the patient or between the transverse muscle and the rectus muscle. The electromechanical actuation device 208 can be implanted in the abdomen at a selected location determined by a clinician. The sterile saline solution 206 can be pumped from the reservoir 204 into the chambers of the cylinders 202 via tubing 210, 212 with the actuation device 208. For example, the actuation device 208 may include electromechanical pumps and valves within the fluid circuit. The cylinders 202 replace non-functioning or limited-functioning erectile tissue in the penis, and the cylinders 202 inflated with the saline solution 206 produce an erection. After sexual intercourse, the actuation device 208 releases the saline solution 206 in the cylinders 202, and the fluid is returned to the reservoir 204 via the tubing and fluidic circuit within the actuation device 208.

In another example of implantable medical device incorporating the features of an electromechanical actuation device incorporating features of medical device 30, the inflatable member 54 can correspond with an inflatable cuff, which may be implemented as an artificial urinary sphincter. In some embodiments, the inflatable cuff is disposed about a urethra at a location proximate the bladder. The actuation device is applied to pump fluid from a reservoir to expand the cuff and to close the urethra. The cuff is deflated to allow a patient to void the bladder. The disclosure describes features of the medical device 30 with reference to a penile prosthesis, such as medical device 200, for illustration, and the features of the medical device 30 can apply to other implementations and components.

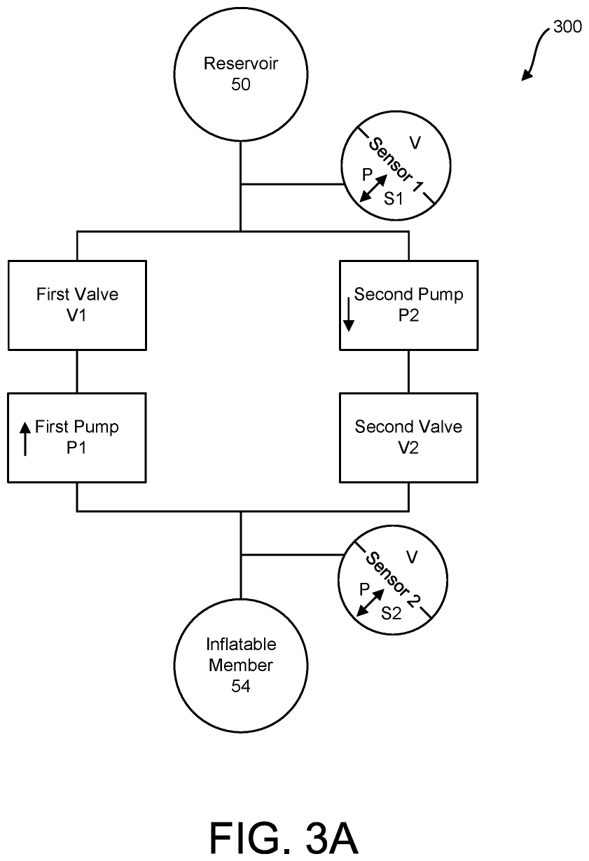

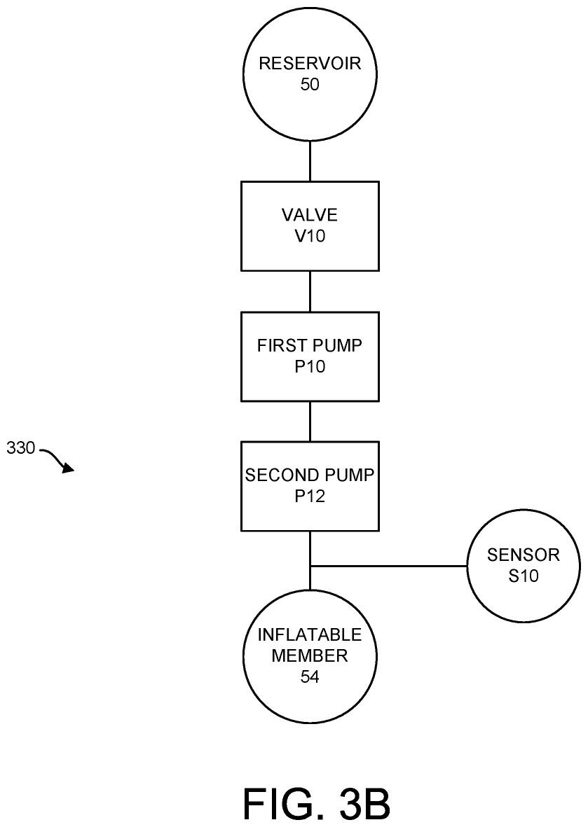

FIG. 3A illustrates an example fluidic architecture 300 for implantable medical device 30, and like parts are labeled with like reference numerals. The example fluidic architecture 300 includes channels to guide the flow of fluid 52 between the reservoir 50 and the inflatable member 54, such as cylinders 202. A first valve V1 in a first fluidic channel controls the flow of fluid 52, generated by a first pumping device P1, from the inflatable member 54 to the reservoir 50. A second valve V2 in a second fluidic channel controls the flow of fluid 52, generated by a second pumping device P2, from the reservoir 50 to the inflatable member 54. In the example, a first pressure sensor S1 senses a fluid pressure at the reservoir 50, and a second pressure sensor S2 senses a fluid pressure at the inflatable member 54. The first and second pressure sensors S1, S2 provide for the monitoring of fluid flow and fluid pressure in the fluidic channels.

In the arrangement of fluid architecture 300, one of the first pump P1 and the second pump P2 is active, while the other of the first pump P1 and the second pump P2 is in a standby mode, such that the first pump and the second pump do not typically operate simultaneously. For example, operation of the first pump P1, with the second pump P2 in the standby mode, provides for the deflation of the inflatable member 54, and operation of the second pump P2, with the first pump P1 in the standby mode, provides for the inflation of the inflatable member 54.

The valves V1, V2 provide for the selective sealing of the respective fluidic channel or channels to maintain a set state of the implantable medical device 30. In some implementations, the valves V1, V2 facilitates the transition between states, i.e., inflated and deflated states, of the implanted medical device 30. For example, selective sealing of the respective fluidic channel or channels by the valves V1, V2 maintain an inflated state or a deflated state of the inflatable member 54. Interaction with the valves V1, V2, and the corresponding change in fluid flow through the fluidic architecture 300 of the implantable medical device 30, may change the set state of the implantable medical device 30. In the illustrated example, the valves V1, V2 are disposed downstream of the respective pumps P1, P2. In an alternative configuration, the order of the valves V1, V2 and respective pumps P1, P2 can be changes such that the pumps P1, P2 are downstream of the respective valves V1, V2.

FIG. 3B illustrates another example fluidic architecture 330 for implantable medical device 30, and like parts are labeled with like reference numerals. The example fluidic architecture 330 includes a channel to guide the flow of fluid 52 between the reservoir 50 and the inflatable member 54, such as cylinders 202. In one example, the cylinders 202 may be coupled to Y-connect tubing that is in fluid communication with the channel. A valve V10 in the fluidic channel controls the flow of fluid 52, generated by a first pumping device P10 and a second pumping device P12, from the reservoir 50 to the inflatable member 54. In the example, a pressure sensor S10 senses a fluid pressure at the inflatable member 54. The pressure sensor S10 provides for the monitoring of fluid flow and fluid pressure in the fluidic channel.

In the arrangement of fluid architecture 330, the first pump P10 and the second pump P12 are simultaneously active or simultaneously inactive, such as in standby mode, such that the first pump P10 and the second pump P12 typically operate together. For example, operation of the first pump P10 and the second pump P12 provide for the inflation of the inflatable member 54. The first pump P10 and the second pump P12 do not operate, and are inactive and in standby mode, for the deflation of the inflatable member 54.

The valve V10 provides for the selective sealing of the fluidic channel to maintain a set state of the implantable medical device 30. In some implementations, the valve V10 facilitates the transition between states, i.e., inflated and deflated states, of the implanted medical device 30. For example, selective sealing of the fluidic channel by the valve V10 maintains an inflated state or a deflated state of the inflatable member 54. Interaction with the valve V10 the corresponding change in fluid flow through the fluidic architecture 330 of the implantable medical device 30, may change the set state of the implantable medical device 30. The order of the fluid components such as valve V10, pumps P10, P12, and pressure senor S10, can be rearrange in the fluidic channel to suit various considerations.

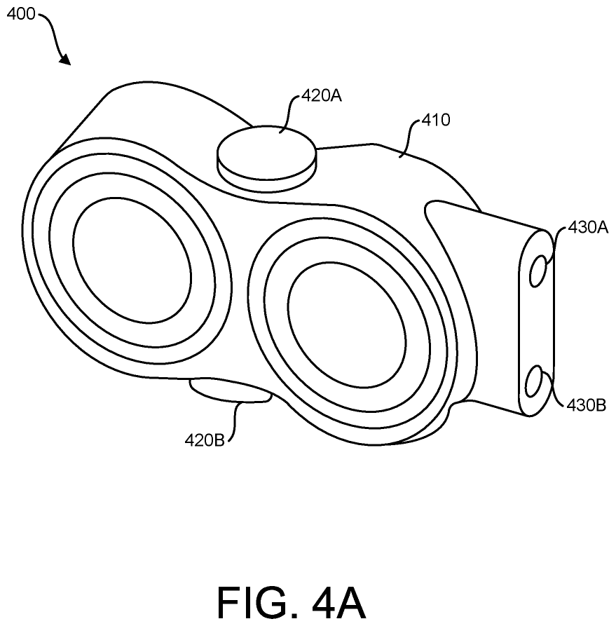

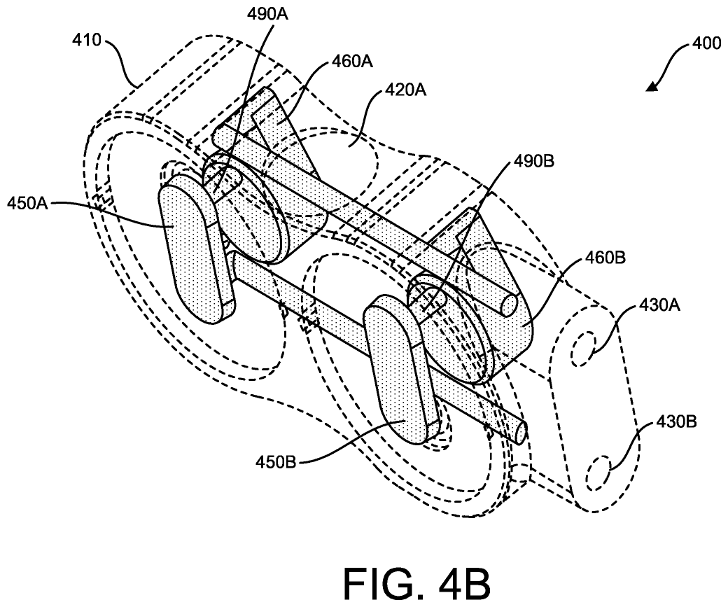

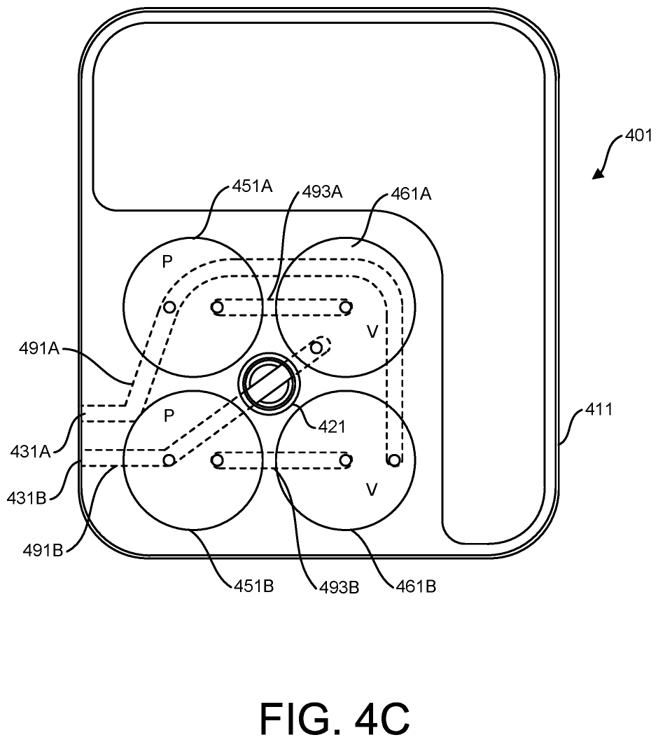

FIGS. 4A and 4B illustrate an example manifold 400 for use with a fluidic circuit 46 of an implantable medical device 30. A manifold in the fluidics circuit 46 can employ a fluidic architecture, such as fluidic architecture 300 or 330, to provide for the controlled transfer and monitoring of fluid in the implantable medical device 30 between the fluid reservoir 50 and the inflatable member 54. The example manifold 400 is illustrated as employing fluidic architecture 300. Manifold 400 can be referred to as a stacked manifold or in a stacked configuration, such as when fluidic components are in a plurality of planes of the manifold.

The manifold 400 includes a housing 410. In FIG. 4B, the housing 410 of the example manifold 400 is in phantom, so that an arrangement of internal fluid passageways and fluidics components such as valves, pumps, and sensors of the manifold 400 are illustrated. Fluid passageways are defined within the housing 410, with fluidics components positioned within the fluid passageways. In some examples, the housing 410 may be manufactured from a solid piece of material. In some examples, the housing 410 is molded, for example, injection molded. In some examples, the housing 410 is made of a metal material such as, for example, titanium, steel, or other biocompatible material. This can allow fluidics components to be installed in fluid passageways defined within the housing 410, and the fluid passageways to be sealed. The manifold 400 or housing 410 manufactured in this manner can be hermetic, such that fluids flowing through the manifold 400 and components received in the manifold 400 are contained within the manifold 400. In a situation in which one or more of the fluidics components includes a non-biocompatible material, the hermetic nature of the manifold 400 can prevent or reduce the likelihood of leaching of these materials into the body of the patient.

In the example illustrated in FIG. 4B, the manifold 400 includes a first pump 450A in fluidic communication with a first valve 460A via a first fluid passageway 490A, and a second pump 450B in fluidic communication with a second valve 460B via a second fluid passageway 490B. The first pump 450A and the first valve 460A direct fluid out of the manifold 400 through a first outlet port 430A, such as to the fluidically coupled reservoir 50. The second pump 450B and the second valve 460B direct fluid out of the manifold 400 through a second outlet port 430B to the fluidically coupled inflatable member 54. A first pressure sensor 420A senses a fluid pressure of fluid flowing between the manifold 400 and the reservoir 50. A second pressure sensor 420B senses a fluid pressure of fluid flowing between the manifold 400 and the inflatable member 54.

In this example, the first pump 450A and second pump 450B are coplanar in a first plane, and the first valve 460A and second valve 460B are coplanar in a second plane in which the first plane is parallel to the second plane of the manifold. For example, the first pump 450A and first valve 460A are opposite each other on the manifold 400, and the second pump 450B and second valve 460B are opposite each other on the manifold 400.

In an example, the first valve 460A and the second valve 460B are normally open valves. In an arrangement in which the first and second valves 460A, 460B are normally open valves, the second valve 460B is actuated to cause the second valve 460B to close while the first pump 450A operates to cause fluid to flow from the manifold 400 to the reservoir 50. Similarly, the first valve 460A is actuated to cause the first valve 460A to close while the second pump 450B operates to cause fluid to flow from the manifold 400 to the inflatable member 54. Normally open valves can provide for the relief of pressure at the inflatable member 54 in the event of faults, failures, or blockages within the fluidics architecture.

FIG. 4C illustrates an example manifold 401 for use with a fluidic circuit 46 of an implantable medical device 30. The example manifold 401 is illustrated as employing fluidic architecture 330. Manifold 401 can be referred to as a planer manifold or in a planer configuration, such as when fluidic components are in a single plane of the manifold.

The manifold 401 includes a housing 411. Fluid passageways are be defined within the housing 411, with fluidics components positioned atop the fluid passageways. In some examples, the housing 411 may be manufactured from a solid piece of material. In some examples, the housing 411 is molded, for example, injection molded. In some examples, the housing 411 is made of a metal material such as, for example, titanium, steel, or other biocompatible material. This can allow fluidics components to be installed in fluid passageways defined within the housing 411, and the fluid passageways to be sealed. The manifold 401 or housing 411 manufactured in this manner can be hermetic, such that fluids flowing through the manifold 401 and components received in the manifold 401 are contained within the manifold 401. In a situation in which one or more of the fluidics components includes a non-biocompatible material, the hermetic nature of the manifold 401 can prevent or reduce the likelihood of leaching of these materials into the body of the patient.

In the example illustrated in FIG. 4C, the manifold 401 includes a first port 431A in fluid communication with first fluid passageway 491A. A first pump 451A is in fluid communication with the first fluid passageway 491A. The first pump 451A is in fluidic communication with a first valve 461A via a second fluid passageway 493A. The manifold 401 also includes a second port 431B in fluid communication with third fluid passageway 491B. A second pump 451B is in fluidic communication with the third fluid passageway 491B. The second pump 451B is in fluid communication with a second valve 461B via a fourth fluid passageway 493B. The first pump 451A and the open first valve 461A direct fluid from first outlet port 431A out of the manifold 401 through a second outlet port 431B, via first fluid passageway 491A to second fluid passageway 493A and via third fluid passageway 491B with a bypass of second pump 451B. The second valve 461B is closed. The second pump 451B and the open second valve 461B direct fluid from second outlet port 431B out of the manifold 401 through first outlet port 431A via third fluid passageway 491B and via fourth fluid passageway 493B with a bypass of first pump 451A in first fluid passageway 491A. The first valve 461B is closed. A pressure sensor 421 in third fluid passageway 491B senses a fluid pressure of fluid flowing between the manifold 401 and the reservoir 50.

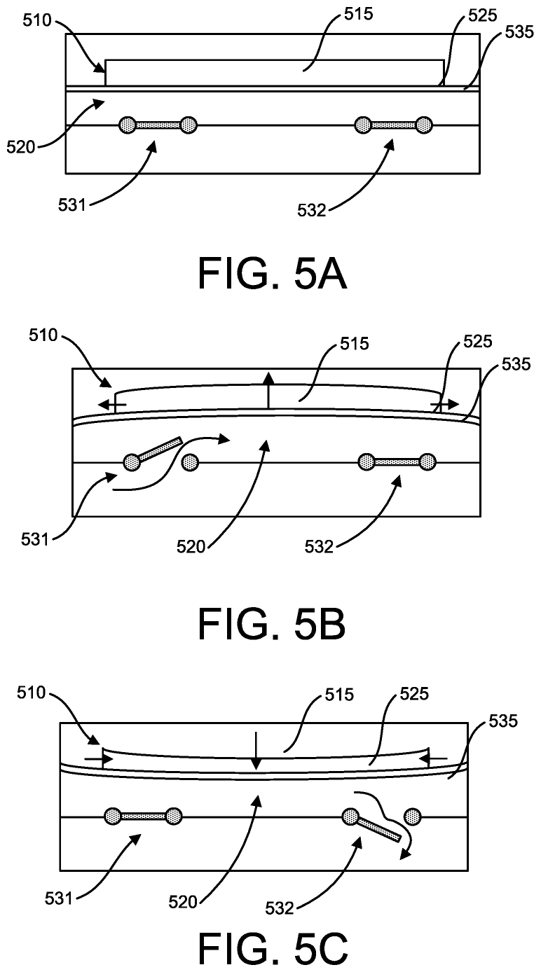

FIGS. 5A to 5C schematically illustrate operation of a pump, such as piezoelectric pump assembly 500, that can be employed in a fluidic architecture, such as fluidic architectures 300 or 330, and can be employed in a stacked manifold configuration such as manifold 400. Additionally, the piezo electric pump assembly 500 can be employed in a planar manifold in which fluid components such as pumps, valves, and pressure sensors are deployed in a plane.

FIG. 5A illustrates a piezoelectric diaphragm 510 positioned in a fluid chamber 520 of a piezoelectric diaphragm pumping device that can provide for the pumping of fluid and the sensing of pressure. In this example, the piezoelectric diaphragm 510 is positioned along an edge portion of the chamber 520 and includes a single layer disc 515 made of a piezoelectric material (for example, a piezo-ceramic disc) mounted on a plate 525 or membrane 525 attached to an insulative diaphragm 535. A first check valve 531 is positioned at a first side of the chamber 520, for example, an inlet end of the chamber 520, corresponding to a first end portion of the piezoelectric diaphragm 510, regulating flow through the chamber 520 in a first direction. A second check valve 532 is positioned at a second side of the chamber 520, for example, an outlet end of the chamber 520, corresponding to a second end portion of the piezoelectric diaphragm 510, regulating flow through the chamber 520 in a second direction. Application of a voltage, or an increase in voltage, causes deformation of the piezo-ceramic disc 515 and a corresponding upstroke of the membrane 525 and diaphragm 535, as illustrated in FIG. 5B. The upstroke of the disc 515 corresponding to a supply stroke draws fluid into the chamber 520 through the first check valve 531 to fill the chamber 520. Release of the voltage, or a decrease in voltage, causes deformation of the disc 515 and a corresponding down stroke, as illustrated in FIG. 5C. This down stroke of the disc 515 corresponding to a pressure stroke displaces fluid out of or expels fluid from the chamber 520 through the second check valve 532. This pumping cycle can be repeated to continue to pump fluid into and out of, or through, the chamber 520.

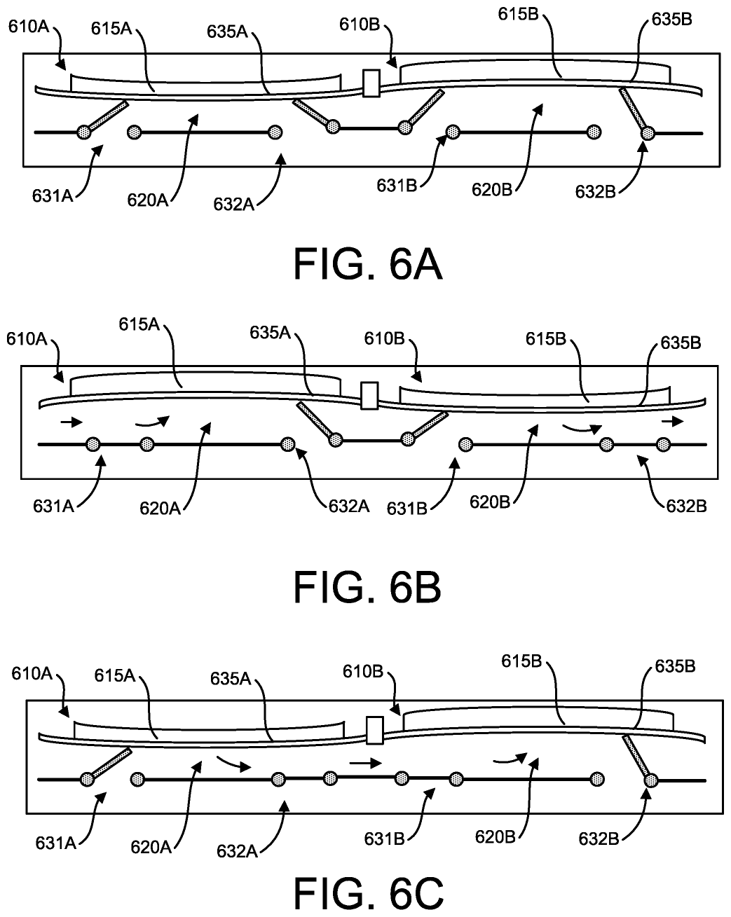

FIGS. 6A to 6C schematically illustrate operation of a dual piezoelectric pump and valve manifold device 600. In particular, FIGS. 6A to 6C illustrate operation of a dual piezoelectric pump and valve device 600 through first, second and third phases of a pumping cycle of fluid through the dual piezoelectric pump and valve device 600. The dual piezoelectric pump and valve manifold device 600 can be employed in a fluidic architecture, such as fluidic architectures 300 or 330, and can be employed in a stacked manifold configuration such as manifold 400 or in a planar manifold.

In the first phase illustrated in FIG. 6A, a first check valve 631A and a second check valve 632A are in a closed position such that fluid does not flow into or out of a first chamber 620A corresponding to a first piezoelectric diaphragm 610A. Similarly, a first check valve 631B and a second check valve 632B are in a closed position such that fluid does not flow into or out of a second chamber 620B corresponding to a second piezoelectric diaphragm 610B.

In response to an application of voltage, a piezo-ceramic disc 615A and membrane 635A of the first piezoelectric diaphragm 610A perform an upstroke, or supply stroke, and a piezo-ceramic disc 615B and membrane 635B of the second piezoelectric diaphragm 610B perform a downstroke, or pressure stroke, from the respective first phase positions shown in FIG. 6A to the respective second phase positions shown in FIG. 6B. Voltage may be applied to the piezo-ceramic disc 615A based on, for example, a fluid pressure and/or a fluid flow rate measured by one of the pressure sensors included in the fluidic architecture described above. Upstroke of the first piezoelectric diaphragm 610A decreases a pressure in the first chamber 620A, opening the first check valve 631A and allowing fluid to flow through the first check valve 631A and into the first chamber 620A, while the second check valve 632A remains closed. Downstroke of the second piezoelectric diaphragm 610B increases a pressure in the second chamber 620B, opening the second check valve 632B and allowing fluid to flow out of the second chamber 620B and through the second check valve 632B, while the first check valve 631B remains closed.

In response to removal of the voltage, the piezo-ceramic disc 615A and membrane 635A of the first piezoelectric diaphragm 610A perform a downstroke, or pressure stroke, and the piezo-ceramic disc 615B and membrane 635B of the second piezoelectric diaphragm 610B perform an upstroke, or supply stroke, from the respective second phase positions shown in FIG. 6B to the respective third phase positions shown in FIG. 6C. Removal of the voltage applied to the piezo-ceramic disc 615A may be based on, for example, a fluid pressure and/or a fluid flow rate measured by one of the pressure sensors included in the fluidic architecture described above. Downstroke of the first piezoelectric diaphragm 610A increases a pressure in the first chamber 620A, closing the first check valve 631A and opening the second check valve 632A, allowing fluid to flow through the second check valve 632A and into the fluid channel toward the second chamber 620B. Upstroke of the second piezoelectric diaphragm 610B decreases a pressure in the second chamber 620B, opening the first check valve 631B and allowing fluid to flow into the second chamber 620B, while the second check valve 632B remains closed.

The first, second and third phases of the pumping cycle of the dual piezoelectric pump and valve device in FIGS. 6A-6C illustrate the refilling of fluid in the first chamber 620A and the discharge of fluid accumulated in the second chamber 620B in going from the first phase (FIG. 6A) to the second phase (FIG. 6B), and the discharge of fluid accumulated in the first chamber 620A and the refilling of fluid into the second chamber 620B in going from the second phase (FIG. 6B) to the third phase (FIG. 6C).

In the example described with respect to FIGS. 6A-6C, the dual piezoelectric pump and valve device includes a first check valve 631A, 631B and a second check valve 632A, 632B respectively associated with the flow through each chamber 620A, 620B. In some implementations, operation of the second check valve 632A of the first chamber 620A and the first check valve 631B of the second chamber 620B can be replaced with a single valve (not shown in FIGS. 6A-6C) that can control the flow between the first chamber 620A and the second chamber 620B in a similar manner to that which is described with respect to FIGS. 6A-6C.

In some examples, a current-mode sensing method may be applied to determine pressure in a piezoelectric diaphragm pump. As current and pressure are linearly interrelated, pressure can be inferred from the amount of current required to move the diaphragm. In this type of current-mode sensing, pressure can be sensed at each pumping cycle as described above, based on the amount of current required to move the diaphragm and fill/empty the respective chamber.

In some examples, an induced-response method may be applied to determine pressure. The induced-response method may make use of the ability of piezoelectric materials to convert movement into voltage (in addition to moving in response to the application of electrical stimulus). As the electro-mechanical actuation and responses of piezoelectric materials are associated with alternating current (AC) signals, the use of the pump as a sensor in, for example, the piezoelectric diaphragm pump, can only measure changes in pressure. In some examples, this can be overcome by controlling an input to one fluid chamber and measuring an output at another fluid chamber. Fluid architecture 330 can be implemented in an example dual piezoelectric pump manifold configuration, such as the example dual piezoelectric pump and valve device shown in FIGS. 6A-6C, having multiple chambers arranged in series. In this example arrangement, the first chamber (for example, the first chamber 620A) may be connected to the second chamber (for example, the second chamber 620B) by a fluid passageway. A known stimulus (i.e., a known voltage level, or a known pulse level) is input at the first chamber, and the output at the second chamber (a voltage level, or a pulse magnitude) is detected. In some examples, a static pressure can be determined based on a known pulse input applied to the first chamber, and the resultant pulse output measured at the second chamber.

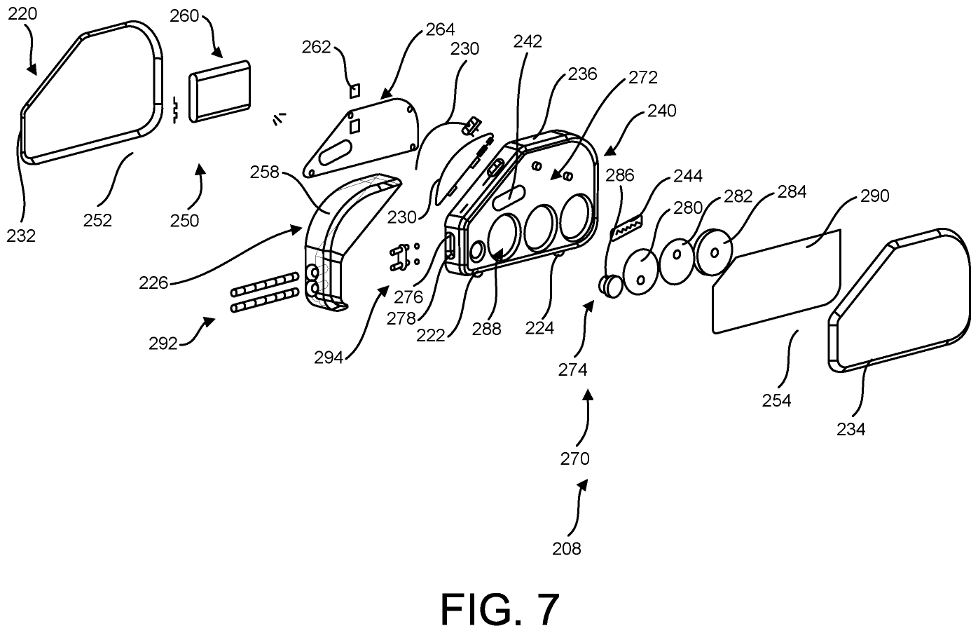

FIG. 7 illustrates an exploded view of the example actuation device 208 of FIG. 2 in which like parts are labeled with like reference numerals. The housing 220 includes a first sidewall 232, a second, opposite side wall 234, a peripheral wall 236, and a frame 240. In the example actuation device 208, the first sidewall 232, second sidewall 234, and peripheral wall 236 are hermetically sealed together to form an internal compartment 250 within the housing 220. The frame 240 is disposed within the internal compartment 250 to form a first partition 252 and a second partition 254 in such a manner that the first partition 252 is hermetically sealed from the second partition 254. In an example, the frame 240 can be integrally formed with the peripheral wall 236 or the first or second sidewall 232, 234. In another example, the frame 240 is welded to the peripheral wall 236 or welded to the first or second sidewall 232, 234. In the illustrated example, the first side wall 232, peripheral wall 236, and frame 240 form the first partition 252. The second side wall 234, peripheral wall 236, and frame 240 form the second partition 254, which is opposite the frame 240 from the first partition 252.

The actuation device 208 can include a header 226 attached to the housing 220 to form an internal region 258 between an inner surface of the header 226 and an outer surface of the housing 220 that includes power and communication interface structures such as the secondary coil 228 and the antenna 230 external to the hermetically scaled housing 220. The header 226 is configured from a dielectric or insulative material, such as a radome, to allow the transmission of power and communication signals between the antenna 230 and a handset programmer or charger, and between the secondary coil 228 and the charger. For example, the header 226 may be comprised of an overmolded polymer affixed to the housing 220 and including the secondary coil 228 and antenna within the internal region 258. In this example, the secondary coil 228 and antenna 230 are constructed from a biocompatible material. In one instance, the secondary coil 228 and antenna 230 can be formed as a coil from a stamped titanium core clad with gold or silver. In another instance, the secondary coil 228 and antenna 230 can be formed from a gold wire.

The actuation device 208 includes an energy storage system, such as a rechargeable power source 260 or a rechargeable battery, and electronic components 262 within the first partition 252. The electronic components 262 can be disposed on a circuit board 264, such as a plurality of circuit boards, within the first partition 252. The rechargeable battery 260 can assume various forms appropriate to provide power for generating desired electrical signals and to store power provided from the electronic components 262. For example, the battery 260 can incorporate lithium-ion (Li+) chemistry, i.e., a lithium-ion battery to operate the electronic components 262. In one example, the electronics components 262 can be implemented by various components including resistors, capacitors, transistors, and integrated circuits disposed on the circuit board 264. The secondary coil 228 and antenna 230 are electrically coupled to the electronic components 262 within the first partition 252, such as via a hermetic feedthrough component.

The electronic components 262 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as Bluetooth Low Energy, which is compatible with several operating systems that can be applied in mobile devices configured as handset programmers. Communication system can include an integrated circuit to implement an applied communication technology. In some examples, the communication system can be used to transmit communication signals to other devices, such as a charger or the handheld programmer, and the communication system can be implemented to generate communication signals and provide the communication signals to the antenna 230 for transmission. In some examples, the communication system can be configured to receive and transmit radiofrequency signals via the antenna 230. The controller can include a microcontroller to operate the recharge system and to receive and operate in response to communication signals or generate communication signals from communication system.

The actuation device 208 also includes a fluidics circuit 270 within the second partition 254 and opposite the frame 240 from the battery 260 and electronic components 262. In the example, the frame 240 can include an opening 242 that includes a hermetic interface 244, such as a feedthrough hermetically affixed to the frame. The electronic components 262 are operably coupled to the fluidics circuit 270 across the frame 240 via the hermetic interface 244. For example, the controller of the electronic components 262, powered by the battery 260, can cause the operation of the fluidics circuit 270 such as to control and monitor the fluidics circuit 270. The fluidics circuit 270 includes a manifold 272 and fluid components 274 operably coupled to the manifold 272. In the illustrated example, the manifold 272 is a structure integrated into the frame 240 such that the manifold 272 and frame 240 together form the hermetic barrier between the first partition 252 and the second partition 254 of the internal compartment 250. For instance, the battery 260, the circuit board 264, or electronic components 262 can be coupled to a first major surface of the manifold 272 in the first partition 252, and the fluidics components 274 are operably coupled to a second, and opposite major surface of the manifold 272 in the second partition 254.

The fluidics circuit 270 is arranged in a fluidic architecture, such as fluidic architecture 330 of FIG. 3B in the illustrated example. The fluidics circuit 270 provides for the transfer of the fluid 206 between the reservoir 204 and the inflatable member such as cylinders 202. The manifold 272, which can be a hermetic manifold, segments and contains the fluid from the internal compartment 250 to reduce the chance of fluid exchange and directs the fluid from a first port 276 to a second port 278 via internal fluid passageways or channels. In the illustrated example, the fluidics components 274 include a plurality of fluid pumps, such as pumps 280, 282, a valve 284 mounted into the manifold 272 in fluidic communication with a manifold passageway to transfer fluid from the first port 276 to the second port 278. In the example of fluid architecture 330, the pumps and valve are in fluid communication with a single fluid passageway between ports 276, 278. The fluidics components 274 also includes a pressure sensor 286 operably coupled to the manifold 272 and in fluidic communication with the passageway to detect a pressure of the fluid within the manifold 272. As indicated, the fluidics components 274 are included in a planar configuration on the manifold 272 in which the pumps 280, 282, valve 284, and pressure sensor 286 are mounted into the manifold 272 on a plane for slim profile within the second partition 254. The manifold 272 can include chambers 288 formed into the second major surface in which the chambers are fluidically coupled to the single passageway within the manifold 272. The chambers are configured to receive the pumps 280, 282, and valve 284 and the pressure sensor 286. The manifold 272 can receive a piezoelectric pump, such as a pump illustrated in FIG. 5A-5C or 6A-6C. The manifold 272 can receive a components cover 290 over the fluidic components 274, which can be hermetically sealed to the second major surface.

The example actuation device 208 can include kink resistant tubing 292 that can extend through the header 226 and attached to the ports 276, 278 via components such as a barb 294 and O-rings. The kink resistant tubing 292 can be attached to the tubing 210, 212 to fluidically couple the actuation device 208 to the reservoir 204 and the inflatable member, such as the cylinders 202.

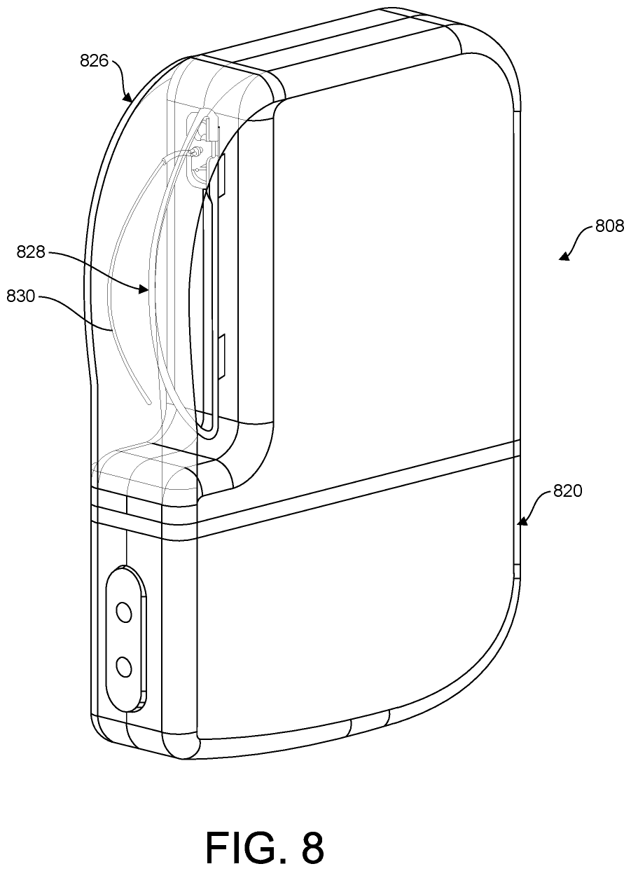

FIG. 8 illustrates another actuation device 808, which may be incorporated into the implantable medical device 200 of FIG. 2 instead of actuation device 208. For instance, the alternate implantable medical device can include an inflatable member, such as a pair of inflatable cylinders 202, a reservoir 204 that may be filled with a fluid, such as a sterile saline solution 206, and electromechanical actuation device 808 in a closed system. The reservoir 204 is fluidically coupled to the actuation device 808 via tubing 210, and the actuation device 808 is fluidically coupled to the cylinders 202 via tubing 212.

The actuation device 808 includes a hermetically sealed housing 820 formed of a biocompatible material such as titanium or steel. In one example, the housing 820 is formed via a plurality of walls welded together. The actuation device 808 includes an internal fluidics circuit to fluidically couple the reservoir 204 to the cylinders 202 via tubing 210, 212. Internal electronic components, powered by a rechargeable power source, can provide for the monitoring and control of various operations of the fluidics circuit such as the transfer of fluid 206 between the reservoir 204 and the cylinders 202. In one example, the electromechanical actuation device 808 can correspond with implantable medical device 30 of FIG. 1. The housing 820 forms an internal compartment defined by the space within the actuation device 808 that is bordered by the plurality of walls. The actuation device 808 can include a header 826 to form a region that includes power and communication interface structures such as a secondary coil 828 and an antenna 830. The header 826 is configured to allow the transmission of power and communication signals between the antenna 830 and a handset programmer or charger, and between the secondary coil 828 and the charger.

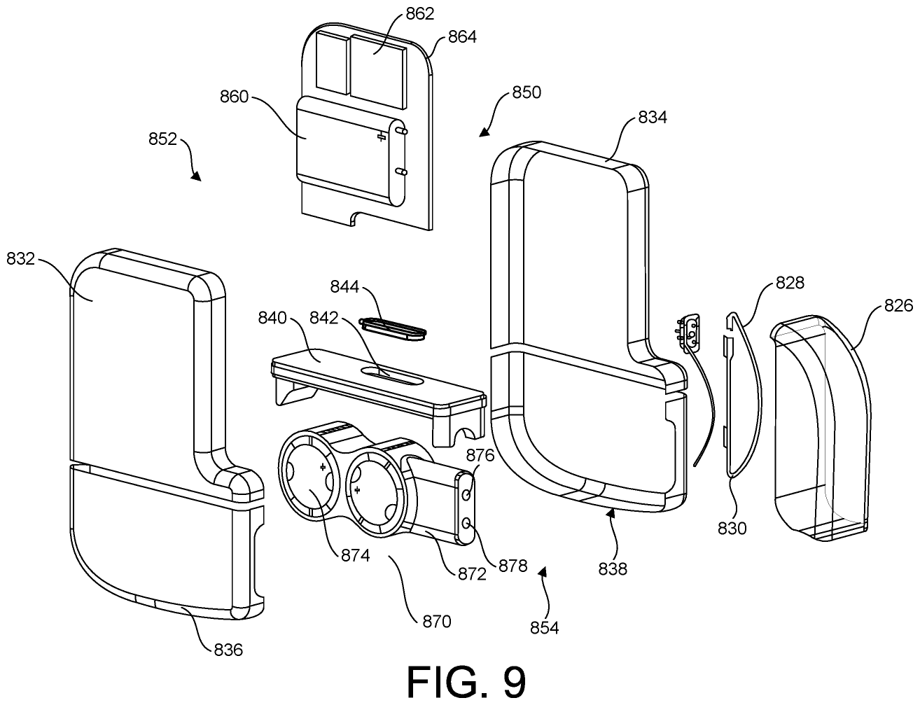

FIG. 9 illustrates an exploded view of the example actuation device 808 of FIG. 8 in which like parts are labeled with like reference numerals. The housing 820 includes a first side wall 832, a second, opposite side wall 834, a third sidewall 836 (adjacent to first side wall 832), a fourth side wall 838 (opposite to the third side wall 836), and a frame 840. In the example actuation device 808, the side walls 832, 834, 836, and 838 are hermetically sealed together to form an internal compartment 850 within the housing 820. The frame 840 is disposed within the internal compartment 850 to form a first partition 852 and a second partition 854 in such a manner that the first partition 852 is hermetically sealed from the second partition 854. In an example, the frame 840 is welded to the side wall 832, 834, 836, 838. In the illustrated example, the first side wall 832, second side wall 834, and frame 240 form the first partition 852. The third side wall 236, fourth side wall 838, and frame 240 form the second partition 854, which is opposite the frame 840 from the first partition 852.

The actuation device 808 can include header 826 attached to the housing 820 to form an internal region 858 between an inner surface of the header 826 and an outer surface of the housing 820 that includes the secondary coil 828 and an antenna 830 external to the hermetically sealed housing 820 in the internal region 858. The header 826 is configured from a dielectric or insulative material, such as a radome, to allow the transmission of power and communication signals between the antenna 830 and a handset programmer or charger, and between the secondary coil 828 and the charger. In this example, the secondary coil 828 and antenna 830 are constructed from a biocompatible material.

The actuation device 808 includes an energy storage system, such as a rechargeable battery 860, and electronic components 862 within the first partition 852. The electronic components 862 can be disposed on a circuit board 864, such as a plurality of circuit boards, within the first partition 852. The secondary coil 828 and antenna 830 are electrically coupled to the electronic components 862 within the first partition 852, such as via a hermetic feedthrough component. The electronic components 862 can include a recharge system, a communication system, and a controller. The recharge system includes hardware configured to interface with the secondary coil 828 to receive power signals, and to provide the power signals in a form suitable to recharge the battery 860 and can include circuitry to reduce the likelihood of overcharging the battery 860. The communication system includes hardware configured to interface with the antenna 830 to receive electrical communication signals. In some examples, the communication system can be used to transmit communication signals to other devices, such as a charger or the handheld programmer, and the communication system can be implemented to generate communication signals and provide the communication signals to the antenna 830 for transmission. The controller can include a microcontroller to operate the recharge system and to receive and operate in response to communication signals or generate communication signals from communication system.

In an alternative example, the actuation device 208, 808 can be constructed without a header 226, 826 respectively, and the interface components of a secondary coil 228, 828 or antenna 230, 830 disposed within the first partition 252, 852 with electronic components 262, 862. In this example, a side wall of the housing 220, 820, can be manufactured to include a hermetically sealed, non-conductive window. An example of such a window is set forth in U.S. patent application Ser. No. 17/951,561 to Maile, et al. titled “Device with Window for Wireless Power Transfer for Urology Implantable Devices,” filed Sep. 23, 2022, which is incorporated by reference into this disclosure to the extent it is not inconsistent with this disclosure. Alternatively, an actuation device may be constructed to not employ outside communication or rechargeable features, and not include a secondary coil and antenna.

The actuation device 808 also includes a fluidics circuit 870 within the second partition 854 and opposite the frame 840 from the battery 860 and electronic components 862. In the example, the frame 840 can include an opening 842 that includes a hermetic interface 844, such as a feedthrough hermetically affixed to the frame. The electronic components 862 are operably coupled to the fluidics circuit 870 across the frame 840 via the hermetic interface 844. For example, the controller of the electronic components 862, powered by the battery 860, can cause the operation of the fluidics circuit 870 such as to control and monitor the fluidics circuit 870. The fluidics circuit 870 includes a manifold 872 and fluid components 874 operably coupled to the manifold 872. The fluidics circuit 870 is arranged in a fluidic architecture, such as fluidic architecture 300 of FIG. 3A in the illustrated example. The fluidics circuit 870 provides for the transfer of the fluid 206 between the reservoir 204 and the inflatable member such as cylinders 202. The manifold 872, which can be a hermetic manifold, segments and contains the fluid from the internal compartment 850 to reduce the chance of fluid exchange and directs the fluid from a first port 876 to a second port 878 via internal fluid passageways or channels. In the illustrated example, the manifold 872 and fluidics components 874 are arranged in the configuration of manifold 400 of FIGS. 4A and 4B.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An implantable medical device, comprising:
a housing forming an internal compartment, the housing including a frame disposed within the internal compartment forming a first partition and a second partition within the internal compartment, the first partition hermetically sealed from the second partition;
at least one electronic component and a power source disposed within the first partition; and
a fluidics circuit including a manifold operably coupled to at least one fluidics component, the fluidics circuit operably coupled to the at least one electronic component across the frame.

2. The implantable medical device of claim 1, wherein the power source is a rechargeable power source including a battery.

3. The implantable medical device of claim 2, wherein the electronic components include a communication system and a recharge system, the recharge system coupled to the power source.

4. The implantable medical device of claim 3, and further comprising an antenna operably coupled to the communication system and a secondary coil operably coupled to the recharge system.

5. The implantable medical device of claim 4, wherein the antenna and secondary coil are disposed outside of the internal compartment, wherein a header is operably coupled to the housing outside of the internal compartment and forming an internal region, and the antenna and secondary coil are disposed within the internal region.

6. The implantable medical device of claim 1, wherein the manifold is integrally formed with the frame.

7. The implantable medical device of claim 6, wherein the manifold is hermetically sealed to the housing to form the first partition and the second partition.

8. The implantable medical device of claim 7, wherein the at least one electronic component is coupled to the manifold in the first partition.

9. The implantable medical device of claim 1, wherein the fluidics circuit is operably coupled to the at least one electronic component across the frame via a hermetically sealed feedthrough connection.

10. The implantable medical device of claim 1, wherein fluidics components are arranged in a planar configuration.

11. The implantable medical device of claim 10, wherein the fluidics components include two piezoelectric pumps in a single fluid channel.

12. The implantable medical device of claim of claim 11, wherein the fluidics components further include a valve and a pressure sensor in the single fluid channel.

13. The implantable medical device of claim 1, wherein the fluidics components are arranged in a stacked configuration.

14. A method of manufacturing an implantable medical system, the method comprising:
forming a frame including a fluidics manifold, the fluidics manifold having a first major surface and a second major surface;
operably coupling at least one electronic component and a power source to the first major surface;
operably coupling at least one fluidics component to the second major surface to form a fluidic circuit;
attaching a housing to the frame to form an internal compartment, wherein the frame defines a first partition of the internal compartment and a second partition of the internal compartment, the first partition hermetically sealed from the second partition; and
operably coupling the at least one electronic component to the fluidic circuit across the frame.

15. The method of claim 14, and further comprising forming a passageway within the fluidics manifold and chambers in the second major surface, the chambers fluidically coupled to passageway, wherein the at least one fluidics component is operably coupled to the chambers.

16. The method of claim 14, wherein the manifold is integrally formed with the frame.

17. An implantable urological device, comprising:
a reservoir configured to receive a fluid;
an inflatable member in fluid communication with the reservoir;
an implantable medical actuation device, comprising:
a housing forming an internal compartment, the housing including a frame disposed within the internal compartment forming a first partition and a second partition within the internal compartment, the first partition hermetically sealed from the second partition;
at least one electronic component and a power source disposed within the first partition; and
a fluidics circuit including a manifold operably coupled to at least one fluidics component, the fluidics circuit operably coupled to the electronic components across the frame.

18. The implantable urological device of claim 17, wherein the inflatable member includes a plurality of inflatable cylinders in fluid communication with the medical actuation device and the reservoir, the plurality of inflatable cylinders configured to be disposed within a corpora cavernosa of a penis.

19. The implantable urological device of claim 17, wherein the medical instrument is configured to be disposed within a retropubic space.

20. The implantable urological device of claim 17, wherein the manifold is integrally formed with the frame.
